(19) United States
(12) Patent Application Publication
(10) Pub. No.: US 2009/0220997 A1
(43) Pub. Date: Sep. 3, 2009

(54) Stimulating G Protein-Coupled Receptors

(75) Inventors: Laurence J. Miller, Scottsdale, AZ (US); Maoqing Dong, Scottsdale, AZ (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 12/066,555

(22) PCT Filed: Sep. 12, 2006
(86) PCT No.: PCT/US06/35621

§ 371 Date: Sep. 9, 2008

Related U.S. Application Data
(60) Provisional application No. 60/716,445, filed on Sep. 12, 2005.

(51) Int. Cl. G01N 33/53; C07K 1/00; C07K 14/00; C07K 7/00

(52) U.S. Cl. 435/7.21; 530/345; 530/324; 530/326; 530/328; 530/330

(57) ABSTRACT

This document provides methods and materials related to activating GPCRs. For example, methods and materials for activating GPCRs present on cells (e.g., human cell) as well as methods and materials for identifying GPCR agonists are provided.

### CROSS-REFERENCE TO RELATED APPLICATIONS

This application claims the benefit of U.S. Provisional Patent Application Ser. No. 60/716,445, filed Sep. 12, 2005.

### STATEMENT AS TO FEDERALLY SPONSORED RESEARCH

This invention was made with government support under Grant number DK46577 awarded by the National Institute of Diabetes and Digestive and Kidney Diseases (NIDDK) of the National Institutes of Health (NIH). The government has certain rights in the invention.

### BACKGROUND

1. Technical Field

This document relates to methods and materials involved in activating G protein-coupled receptors (GPCRs).

2. Background Information

In general, GPCRs have seven membrane spanning domains linked by three extracellular and three intracellular loops, with an extracellular N-terminus and a cytoplasmic C-terminal tail. Upon ligand binding, these receptors activate G proteins. Before agonist binding to a GPCR, the three subunits of a G protein are bound together. Binding of an agonist causes an interaction between the GPCR and the G protein. The interaction results in dissociation of the α subunit from the βγ subunit complex of the G protein. The separated α and/or βγ subunits may then interact with effectors.

### SUMMARY

This document provides methods and materials related to activating GPCRs. For example, this document provides methods and materials for activating GPCRs present on cells (e.g., human cells) as well as methods and materials for identifying GPCR agonists.

In general, one aspect of this document features a method of stimulating a G protein-coupled receptor. The method comprises, or consists essentially of, contacting the receptor with an agonist, wherein the agonist binds to a region of the receptor that is between the beginning of transmembrane domain 6 and the end of transmembrane domain 7 of the receptor. The G protein-coupled receptor can be a class B G protein-coupled receptor. The G protein-coupled receptor can be a human receptor. The G protein-coupled receptor can be a calcitonin receptor, a vasoactive intestinal peptide receptor 1 (VPAC1 receptor), a glucagon-like peptide 1 receptor, or a secretin receptor. The agonist can be a polypeptide comprising less than 30 amino acids. The agonist can be a polypeptide comprising less than six amino acids. The agonist can be a polypeptide comprising less four amino acids. The agonist can be a cyclic polypeptide. The agonist can contain less than 30 amino acids and contain an amino acid sequence located between the second and third conserved cysteine residues of the G protein-coupled receptor. The region can contain about 10 amino acids. At least a portion of the region can be within the loop between the transmembrane domain 6 and the transmembrane domain 7. The agonist can contain a tryptophan-aspartate-asparagine sequence or a structure that is conformationally comparable to the sequence.

In another aspect, this document features a method for identifying an agonist of a G protein-coupled receptor. The method comprises, or consists essentially of: (a) obtaining a test agent comprising a tryptophan-aspartate-asparagine sequence or a structure that is conformationally comparable to the sequence, (b) contacting a cell comprising a G protein-coupled receptor with the test agent, and (c) determining whether or not the test agent activated the G protein-coupled receptor, wherein the presence of the activation indicates that the test agent is an agonist of the G protein-coupled receptor. The G protein-coupled receptor can be a class B G protein-coupled receptor. The G protein-coupled receptor can be a human receptor. The G protein-coupled receptor can be a calcitonin receptor, a VPAC1 receptor, a glucagon-like peptide 1 receptor, or a secretin receptor. The test agent can be a polypeptide comprising less than 30 amino acids. The test agent can be a polypeptide comprising less than 10 amino acids. The test agent can be a polypeptide comprising less than 5 amino acids. The test agent can be a non-polypeptide molecule. The cell can express a secretin receptor. The cell can be a Chinese hamster ovary cell. The determining step can include a cAMP assay.

In another aspect, this document features a substantially pure polypeptide less than 50 amino acid residues in length, where the polypeptide is capable of binding to a region of a G protein-coupled receptor that is between the beginning of transmembrane domain 6 and the end of transmembrane domain 7 of the receptor. The polypeptide can be less than 40 amino acid residues in length, less than 30 amino acid residues in length, less than 20 amino acid residues in length, less than 10 amino acid residues in length, or less than 5 amino acid residues in length. The polypeptide can comprise an amino acid sequence selected from the group consisting of SEQ ID NO:1, SEQ ID NOs:3-4, SEQ ID NO:6, and SEQ ID NOs:22-32. The polypeptide can be cyclic. The polypeptide can comprise a diaminopropionic acid at the amino terminus and an aspartic acid residue at the carboxyl terminus, where the diaminopropionic acid is linked to the aspartic acid to form a cyclic polypeptide.

In another aspect, this document features a method for identifying an agonist of a G protein-coupled receptor. The method comprises, or consists essentially of: (a) contacting a cell comprising a G protein-coupled receptor lacking an extracellular portion of the amino terminus of the receptor with a test agent, and (b) determining whether or not the test agent activated the G protein-coupled receptor, where the presence of the activation indicates that the test agent is an agonist of the G protein-coupled receptor.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a table listing sequences of polypeptides used in cAMP assays. The sequences of the endogenous polypeptides correspond to regions of the amino terminus of Class B G protein-coupled receptors. The polypeptides were synthesized to have diaminopropionic acid at their amino termini and an Asp residue at their carboxyl termini, with cyclic polypeptides linking these two residues through their side chains. SecR(24-53) (SEQ ID NO:1) is a polypeptide corresponding to amino acids 46 to 75 of the rat secretin receptor (GenBank Accession Number AAH81781; SEQ ID NO:2). LWDNM (SEQ ID NO:3) is a polypeptide corresponding to amino acids 69 to 73 of the rat secretin receptor (GenBank Accession Number AAH81781; SEQ ID NO:2). Bpa refers to p-benzoyl-L-phenylalanine. VPAC1(51-71) (SEQ ID NO:4) is a polypeptide corresponding to amino acids 51 to 71 of the rat VPAC1 receptor (SEQ ID NO:5). CTR(73-80) (SEQ ID NO:6) is a polypeptide corresponding to amino acids 73 to 80 of the human calcitonin receptor (SEQ ID NO:7).

FIG. 2 contains a series of graphs plotting accumulation of cAMP versus concentration of polypeptide used to stimulate cells. Presented in panel A are curves of cAMP accumulation in Chinese hamster ovary cells expressing the secretin receptor (CHO-SecR), and in the cells of the parental CHO cell line not expressing the secretin receptor, in response to increasing concentrations of secretin or a synthetic polypeptide (SEQ ID NO:1) corresponding to the region of the amino terminus of the secretin receptor between the first and third conserved cysteine residues, Cys²⁴ and Cys⁵³ (corresponding to residues 46 to 75 of GenBank Accession Number AAH81781). Presented in panel B are curves of cAMP accumulation in CHO-SecR cells in response to increasing concentrations of the indicated pentapeptide (SEQ ID NO:3), tripeptides, or dipeptides. Presented in panel C are curves of cAMP accumulation in CHO-SecR cells in response to increasing concentrations of a linear tripeptide, cyclic tripeptides, or a myristolated cyclic tripeptide. Adherent cells that were 75 percent confluent were stimulated with the polypeptide in Krebs-Ringers-HEPES medium for 30 min at 37° C. Cellular cAMP was quantified as previously reported (Holtmann et al., J. Biol. Chem. 270, 14394 (1995)). Values represent means±S.E.M. of data from a minimum of three independent assays performed in duplicate. Myr indicates myristoylated cyclic tripeptide.

FIG. 3 contains data that localize the site of action of the endogenous secretin receptor polypeptide. Panel A presents stimulation of cAMP activity in CHO-SecR cells in response to increasing concentrations of photolabile analogues of the secretin receptor polypeptide (WDN-Bpa represents the cyclic WDN polypeptide with Bpa-Tyr on its carboxyl terminus; Bpa-WDN represents the cyclic WDN polypeptide with Tyr-Bpa on its amino terminus). Values represent means±S.E.M. of data from three independent assays performed in duplicate. Panel B illustrates the ability of these radioiodinated photoprobes to covalently label the HA-tagged secretin receptor. Presented is a representative autoradiograph of proteins immunoprecipitated with anti-HA antibody in the absence or presence of competing HA polypeptide and subjected to gel electrophoresis. The migration of the labeled band after deglycosylation with endoglycosidase F (EF) is also shown. Panels C and D illustrate the migration of the labeled band after cyanogen bromide (CNBr) cleavage, as well as the analogous reaction performed with the 1334M mutant secretin receptor. SEQ ID NO:8 sets forth the amino acid sequence of the rat secretin receptor illustrated in Panel C, which does not include the signal sequence. The 22 amino acid signal sequence is set forth in SEQ ID NO:9. SEQ ID NO:10 sets forth the amino acid sequence of the labeled band after cyanogen bromide cleavage illustrated in Panel D. The procedures for photoaffinity labeling, immunoprecipitation, deglycosylation, and polypeptide mapping of ligand binding domains by CNBr cleavage are described elsewhere (Dong et al., J. Biol. Chem. 274, 19161 (1999)).

FIG. 4 contains a series of graphs plotting cAMP accumulation versus concentration of amino-terminal polypeptides from the calcitonin (residues 73-80; SEQ ID NO:6) and VPAC1 (residues 51-71; SEQ ID NO:4) receptors that were used to stimulate cells expressing Class B receptors.

FIG. 5 is an alignment of amino acid sequences of vasoactive intestinal polypeptide receptor 2 precursor (VIPR2_HUMAN; GenBank accession number NP_{—}003373; SEQ ID NO:11), pituitary adenylate cyclase activating polypeptide type I receptor precursor (PACR_HUMAN; GenBank accession number NP_{—}001109; SEQ ID NO:12), vasoactive intestinal polypeptide receptor 1 precursor (VIPR1_HUMAN; GenBank accession number NP_{—}004615; SEQ ID NO:13), secretin receptor precursor (SCTR_HUMAN; GenBank accession number NP_{—}002971; SEQ ID NO:14), glucagon-like peptide 1 receptor precursor (GLP1R_HUMAN; GenBank accession number NP_{—}002053; SEQ ID NO:15), glucagon-like peptide 2 receptor precursor (GLP2R_HUMAN; GenBank accession number NP_{—}004237; SEQ ID NO:16), parathyroid hormone/parathyroid hormone-related peptide receptor precursor (PTHR1_HUMAN; GenBank accession number NP_{—}000307; SEQ ID NO:17), parathyroid hormone receptor precursor (PTHR2_HUMAN; GenBank accession number NP_{—}005039; SEQ ID NO:18), corticotropin releasing factor receptor 2 precursor (CRFR2_HUMAN; GenBank accession number NP_{—}001874; SEQ ID NO:19), corticotropin releasing factor receptor 1 precursor (CRFR1_HUMAN; GenBank accession number NP_{—}004373; SEQ ID NO:20), calcitonin gene-related peptide type 1 receptor precursor (CALRL_HUMAN; GenBank accession number NP_{—}005786; SEQ ID NO:21), and calcitonin receptor precursor (CALCR_HUMAN; GenBank accession number NP_{—}001733; SEQ ID NO:7).

FIG. 6 contains a series of graphs plotting cAMP accumulation versus concentration of the WDN tripeptide or amino-terminal polypeptides from the VPAC1 receptor (residues 51-71; SEQ ID NO:4), the calcitonin receptor (residues 73-80; SEQ ID NO:6), and the GLP1R (residues 63-70; SEQ ID NO:22) that were used to stimulate cells expressing Class B receptors as indicated.

### DETAILED DESCRIPTION

This document provides methods and materials related to activating GPCRs. For example, this document provides methods and materials for activating GPCRs present on cells (e.g., human cell) as well as methods and materials for identifying GPCR agonists. The methods and materials provided herein can be used to activate any type of GPCR. For example, the methods and materials provided herein can be used to activate, without limitation, a vasoactive intestinal polypeptide receptor 2, pituitary adenylate cyclase activating polypeptide type I receptor, vasoactive intestinal polypeptide receptor 1, secretin receptor, glucagon-like peptide 1 receptor, glucagon-like peptide 2 receptor, parathyroid hormone/parathyroid hormone-related peptide receptor, parathyroid hormone receptor, corticotropin releasing factor receptor 2, corticotropin releasing factor receptor 1, calcitonin gene-related peptide type 1 receptor, and calcitonin receptor. Such receptors can be from any species including, without limitation, rats, mice, dogs, cats, horses, cows, pigs, monkeys, and humans.

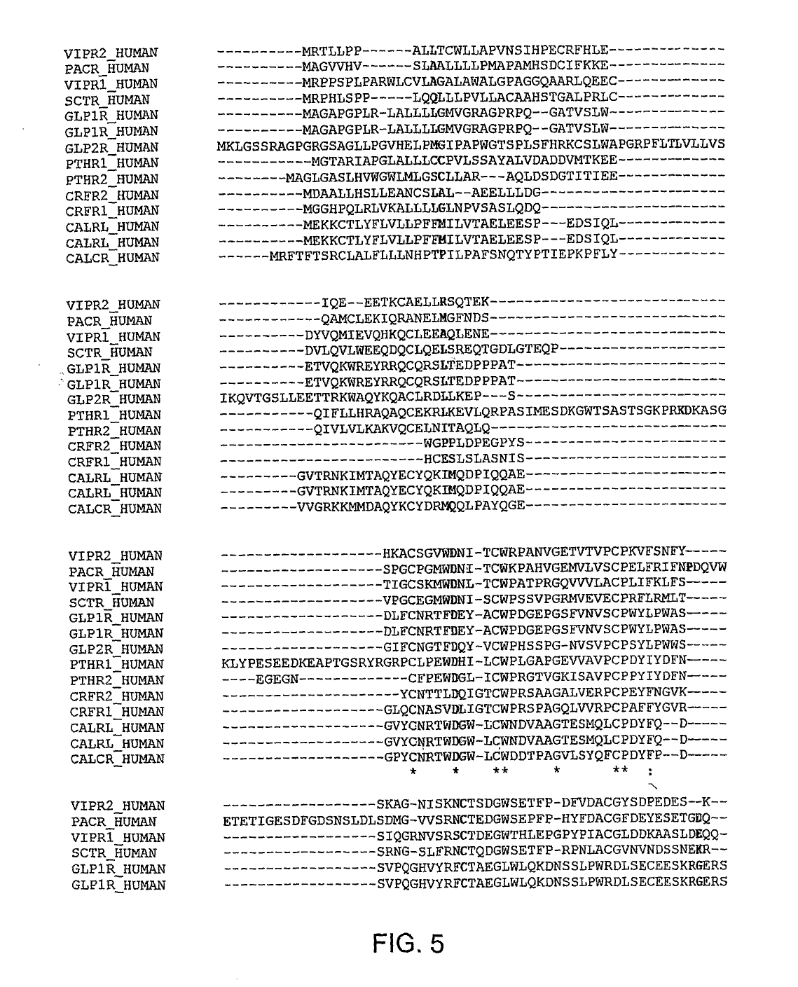

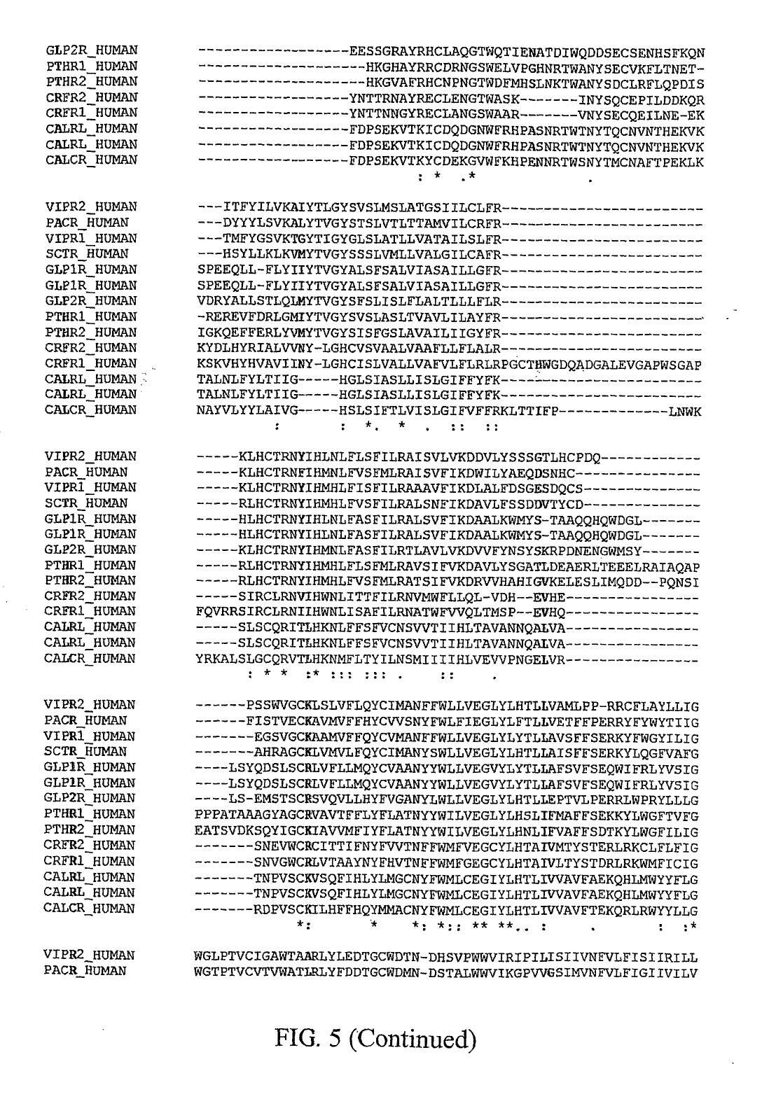

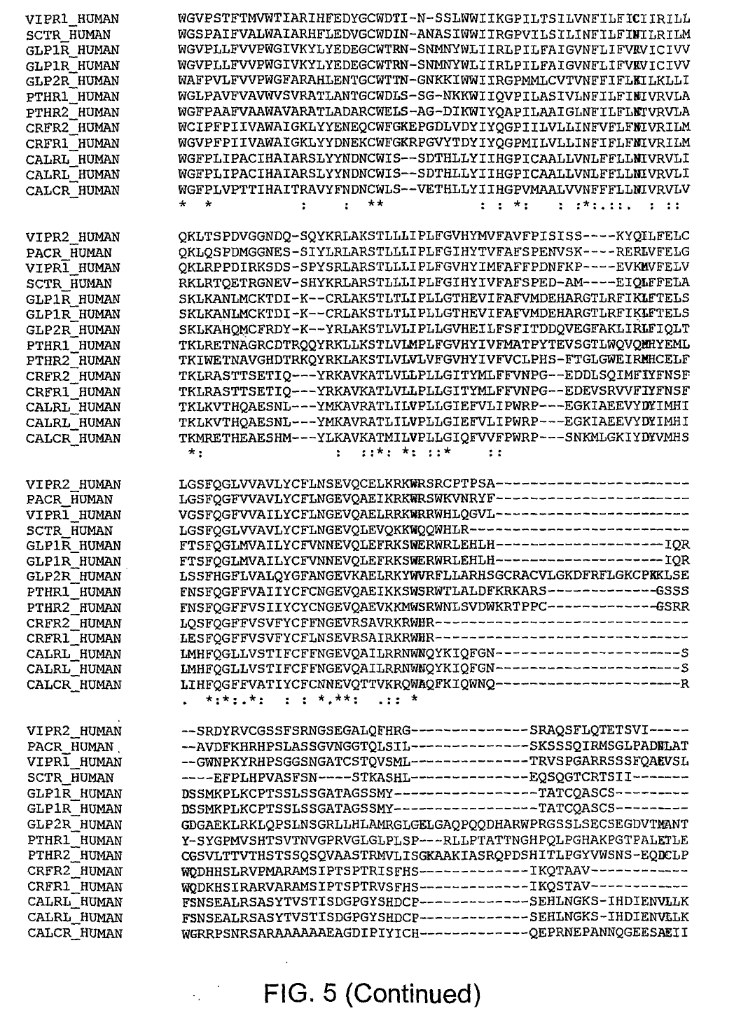

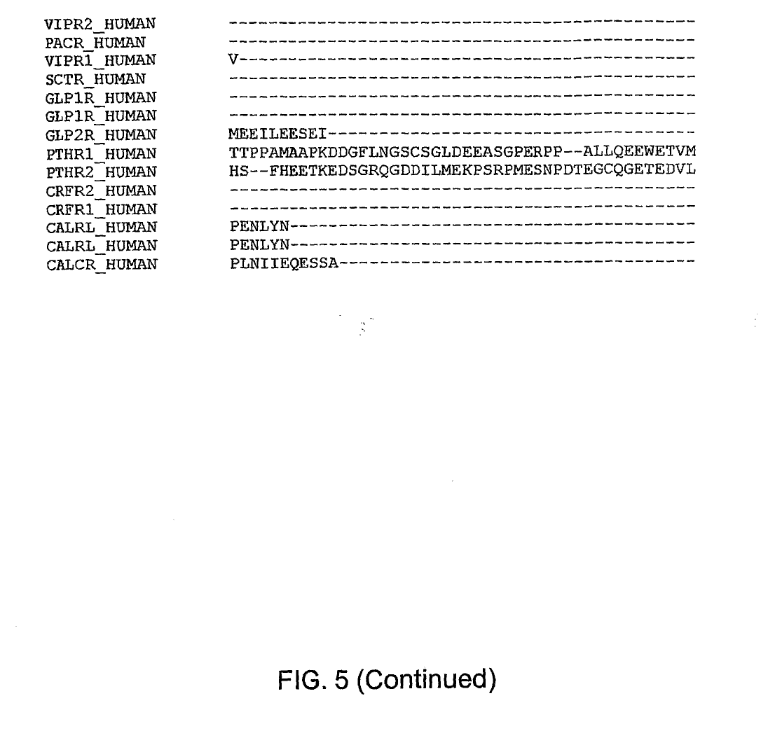

As described herein, agents having the ability to bind to a region of a GPCR that is located between the beginning of transmembrane domain (TMD) 6 and the end of TMD 7 of a GPCR can be used activate GPCRs. In some cases, the region can be the portion of a GPCR that extends from the last few amino acid residues of TMD 6 to the first few amino acid residues of the loop between TMD 6 and 7. An example of such a region can be amino acid residues 357 to 366 of a human secretin receptor precursor (SEQ ID NO:14). Other regions include, without limitation, those regions corresponding to amino acid residues 357 to 366 of human secretin receptor precursor as set forth in FIG. 5. For example, the region can be amino acid residues 325 to 333 of a human CRFR2 (SEQ ID NO:19), amino acid residues 329 to 337 of a human CRFR1 (SEQ ID NO:20), amino acid residues 423 to 433 of a human PTHR1 (SEQ ID NO:17), amino acid residues 378 to 387 of a human PTHR2 (SEQ ID NO:18), amino acids 366 to 376 of a human GLP1R (SEQ ID NO:15), amino acids 400 to 410 of a human GLP2R (SEQ ID NO:16), amino acids 343 to 353 of a human VIPR2 (SEQ ID NO:11), amino acids 368 to 378 of a human PACR (SEQ ID NO:12), amino acids 356 to 366 of a human VIPR1 (SEQ ID NO:13), amino acids 351 to 358 of a human CALRL (SEQ ID NO:21), or amino acids 358 to 365 of a human CALCR (SEQ ID NO:7).

Any type of agent can be used as a GPCR agonist. In some cases, the agent can be a polypeptide having a tryptophan-aspartate-asparagine sequence. In some cases, the agent can be a polypeptide having a tryptophan-aspartate-glycine sequence, a tryptophan-aspartate-histidine sequence, a valine-aspartate-leucine sequence, a leucine-aspartate-glutamine sequence, a phenylalanine-aspartate-glutamine sequence, or a phenylalanine-aspartate-glutamic acid sequence. Such polypeptides can be any length. For example, a polypeptide containing a tryptophan-aspartate-asparagine sequence and having the ability to activate a GPCR can be less than 30 amino acid residues (e.g., less than 25, 24, 23, 22, 21, 20, 19, 18, 17, 16, 15, 14, 13, 12, 11, 10, 9, 8, 7, 6, 5, or 4 amino acid residues) in length.

In some cases, a GPCR agonist can be a polypeptide having five amino acid residues. For example, a GPCR agonist can be a polypeptide having a valine-tryptophan-aspartate-asparagine-isoleucine sequence (SEQ ID NO:23), a methionine-tryptophan-aspartate-asparagine-isoleucine sequence (SEQ ID NO:24), a methionine-tryptophan-aspartate-asparagine-leucine sequence (SEQ ID NO:25), a leucine-tryptophan-aspartate-asparagine-methionine sequence (SEQ ID NO:3), a threonine-phenylalanine-aspartate-glutamic acid-tyrosine sequence (SEQ ID NO:26), a threonine-phenylalanine-aspartate-glutamine-tyrosine sequence (SEQ ID NO:27), a threonine-leucine-aspartate-glutamine-isoleucine sequence (SEQ ID NO:28), a threonine-tryptophan-aspartate-glycine-tryptophan sequence (SEQ ID NO:29), a glutamic acid-tryptophan-aspartate-histidine-isoleucine sequence (SEQ ID NO:30), a glutamic acid-tryptophan-aspartate-glycine-leucine sequence (SEQ ID NO:31), or a serine-valine-aspartate-leucine-isoleucine sequence (SEQ ID NO:32).

In some cases, a GPCR agonist can be a polypeptide containing a region from the GPCR. For example, such a region can be amino acid residues 66 to 75 of a human secretin receptor precursor (SEQ ID NO:14). Other such regions include, without limitation, those regions corresponding to amino acid residues 66 to 75 of human secretin receptor precursor as set forth in FIG. 5. For example such a region can be amino acid residues 40 to 50 of a human CRFR2 (SEQ ID NO:19), amino acid residues 44 to 54 of a human CRFR1 (SEQ ID NO:20), amino acid residues 108 to 117 of a human PTHR1 (SEQ ID NO:17), amino acid residues 63 to 72 of a human PTHR2 (SEQ ID NO:18), 62 to 71 of a human GLP1R (SEQ ID NO:15), amino acid residues 96 to 105 of a human GLP2R (SEQ ID NO:16), amino acid residues 52 to 61 of a human VIPR2 (SEQ ID NO:11), amino acid residues 54 to 63 of a human PACR (SEQ ID NO:12), amino acid residues 63 to 72 of a human VIPR1 (SEQ ID NO:13), amino acid residues 65 to 74 of a human CALRL (SEQ ID NO:21), or amino acid residues 72 to 81 of a human CALCR (SEQ ID NO:7).

In some cases, a GPCR agonist can be a polypeptide containing a region between the first and second conserved cysteine residues of a GPCR. For example, such a region can be amino acid residues 67 to 74 of human secretin receptor precursor (SEQ ID NO:14), amino acid residues 41 to 49 of a human CRFR2 (SEQ ID NO:19), amino acid residues 44 to 53 of a human CRFR1 (SEQ ID NO:20), amino acid residues 109 to 116 of a human PTHR1 (SEQ ID NO:17), amino acid residues 64 to 71 of a human PTHR2 (SEQ ID NO:18), 63 to 70 of a human GLP1R (SEQ ID NO:15), amino acid residues 97 to 104 of a human GLP2R (SEQ ID NO:16), amino acid residues 53 to 60 of a human VIPR2 (SEQ ID NO:11), amino acid residues 55 to 62 of a human PACR (SEQ ID NO:12), amino acid residues 64 to 71 of a human VIPR1 (SEQ ID NO:13), amino acid residues 66 to 73 of a human CALRL (SEQ ID NO:21), or amino acid residues 73 to 80 of a human CALCR (SEQ ID NO:7).

In some cases, a GPCR agonist can be a polypeptide containing a region from the first conserved cysteine residue to the third conserved cysteine of a GPCR. For example, such a region can be amino acid residues 66 to 89 of a human secretin receptor precursor (SEQ ID NO:14). Other such regions include, without limitation, those regions corresponding to amino acids 66 to 89 of a human secretin receptor precursor as set forth in FIG. 5. In some cases, a GPCR agonist can be a polypeptide having at least 90% percent identity to an amino acid sequence of a GPCR agonist polypeptide described above.

As used herein, the term “percent sequence identity” refers to the degree of identity between any given query sequence and a subject sequence. A subject sequence typically has a length that is more than 80%, e.g., more than 82%, 85%, 87%, 89%, 90%, 93%, 95%, 97%, 99%, 100%, 105%, 110%, 115%, or 120%, of the length of the query sequence. A query amino acid sequence can be aligned to one or more subject amino acid sequences using the computer program ClustalW (version 1.83, default parameters), which allows alignments of nucleic acid or polypeptide sequences to be carried out across their entire length (global alignment). Chenna et al., Nucleic Acids Res., 31(13):3497-500 (2003). ClustalW can be run, for example, at the Baylor College of Medicine Search Launcher site (searchlauncher.bcm.tmc.edu/multi-align/multi-align.html) and at the European Bioinformatics Institute site on the World Wide Web (ebi.ac.ukl/clustalw).

ClustalW calculates the best match between a query and one or more subject sequences, and aligns them so that identities, similarities and differences can be determined. Gaps of one or more residues can be inserted into a query sequence, a subject sequence, or both, to maximize sequence alignments. The output is a sequence alignment that reflects the relationship between sequences. To determine a percent identity between a query sequence and a subject sequence, ClustalW divides the number of identities in the best alignment by the number of residues compared (gap positions are excluded), and multiplies the result by 100. The output is the percent identity of the subject sequence with respect to the query sequence.

The term “substantially pure” as used herein with respect to a polypeptide refers to a polypeptide that is substantially free of other polypeptides, lipids, carbohydrates, and nucleic acids with which it is associated in nature. A substantially pure polypeptide can be at least about 60, 65, 70, 75, 80, 85, 90, 95, or 99 percent pure. Typically, a substantially pure polypeptide will yield a single major band on a polyacrylamide gel. Substantially pure polypeptides can be obtained, for example, by extraction from a natural source, chemical synthesis, or by recombinant production in a host cell. To recombinantly produce a polypeptide, a nucleic acid containing a nucleic acid sequence encoding a polypeptide of interest can be ligated into an expression vector and used to transform host cells, e.g., bacterial, insect, yeast, mammalian, or plant cells. The expressed polypeptide can be extracted from the host cells and purified using techniques known to persons having ordinary skill in the art.

In some cases, a GPCR agonist can be a polypeptide containing an additional non-polypeptide component such as a myristoyl group. An example of such a GPCR agonist includes, without limitation, a myristoylated polypeptide (e.g., myristoylated WDN tripeptide). In some cases, a GPCR agonist can be a polypeptide that is covalently attached to oligomers, such as short, amphiphilic oligomers that enable oral administration or improve the pharmacokinetic or pharmacodynamic profile of the conjugated polypeptide. The oligomers can comprise water soluble polyethylene glycol (PEG) and lipid soluble alkyls (short chain fatty acid polymers). See, for example, International Patent Application Publication No. WO 2004/047871. In some cases, a GPCR agonist can be a polypeptide that is fused to the Fc domain of an immunoglobulin molecule (e.g., an IgG1 molecule) such that active transport of the fusion polypeptide across epithelial cell barriers via the Fc receptor occurs.

In some cases, a GPCR agonist can be a cyclic polypeptide. An example of such a GPCR agonist includes, without limitation, cyclic WDN (tryptophan-aspartate-asparagine) tripeptide. In some cases, a GPCR agonist can be a cyclic polypeptide containing a myristoyl group. In some cases, a GPCR agonist can be a small molecule or can contain a structure that is conformationally comparable to a tryptophan-aspartate-asparagine sequence. Any method can be used to obtain molecules having a structure that is conformationally comparable to a tryptophan-aspartate-asparagine sequence including, without limitation, common computer modeling methods.

This document also provides methods and materials related to identifying an agonist of a GPCR. For example, this document provides methods and materials for identifying an agonist of a secretin receptor activity. Agonists of a GPCR can be identified using any method, such as by measuring cAMP accumulation with a cAMP assay. An example of a commercially available cAMP assay kit is the cAMP-Screen® assay (Applied Biosystems, Foster City, Calif. 94404). A cAMP assay can be performed using cells that express a GPCR. Such cells can be generated by transiently or stably transfecting cells with a recombinant nucleic acid construct comprising a sequence encoding a GPCR operably linked to a promoter that drives transcription in the transfected cells. The cells can be obtained from a cell line that does not express the GPCR, or expresses only low levels of the GPCR. A nucleic acid encoding a GPCR can be produced by standard techniques. For example, polymerase chain reaction (PCR) techniques can be used to obtain an isolated nucleic acid encoding a GPCR. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Common techniques (e.g., those described in Sambrook et al., (1989) Molecular Cloning, second edition, Cold Spring harbor Laboratory, Plainview, N.Y. and Current Protocols in Molecular Biology, Ausubel et al., eds., John Wiley & Sons, Inc., 1997) can be used to generate a nucleic acid construct, such as an expression construct, containing a nucleic acid encoding a GPCR. Such techniques also can be used to generate cells expressing the GPCR. Cells expressing a GPCR can be mock-treated or treated with a candidate GPCR agonist. Any effect of a GPCR agonist, such as increased accumulation of cAMP, can be compared in the mock-treated and the candidate GPCR agonist-treated cells to determine whether or not the candidate GPCR agonist is an agonist of the GPCR. For example, an increased cAMP accumulation in the cells treated with the candidate GPCR agonist relative to the mock-treated cells can indicate that the candidate GPCR agonist is an agonist of the GPCR. The dose response and time course of the effect can also be determined. In some cases, candidate GPCR agonists can be tested in vivo, such as in an animal model of diabetes or obesity.

In some embodiments, test agents can be obtained and tested for the ability to activate a GPCR. In such cases, any test agent can be used including, without limitation, polypeptides, modified polypeptides, cyclic polypeptides, and non-polypeptide agents (e.g., small molecules). In some cases, the test agents can be selected to have a tryptophan-aspartate-asparagine sequence or a structure that is conformationally comparable to a tryptophan-aspartate-asparagine sequence. For example, a GPCR agonist can be identified by (a) obtaining a collection of small molecules having a structure that is conformationally comparable to a tryptophan-aspartate-asparagine sequence, (b) contacting cells expressing a GPCR with one or more small molecules from the collection, and (c) determining whether or not one or more of the small molecules activates the GPCR. Small molecules having the ability to activate the GPCR can be classified as being an agonist of that GPCR.

A GPCR agonist can be used to identify additional GPCR agonists. For example, a GPCR agonist (e.g., a polypeptide GPCR agonist described herein) can be labeled (e.g., with a fluorescent fluorophore or with I¹²⁵) and used in a binding assay to identify additional GPCR agonists, such as small molecule GPCR agonists. Binding assays (e.g., competitive or displacement binding assays) can be carried out using cells expressing a GPCR, as described above, or using preparations of membranes from such cells. Large numbers of molecules, such as small molecule libraries, can be screened to identify molecules that displace binding of a labeled GPCR agonist to a GPCR. A molecule that displaces the interaction of another molecule with a GPCR can interact with the GPCR in the same region of the GPCR. Molecules that displace binding of the labeled GPCR agonist can be evaluated for GPCR agonist activity using any method, such as a method described herein (e.g., measurement of cAMP accumulation). In addition, molecules that displace binding of a labeled GPCR agonist can be screened to determine whether or not they displace the binding of other labeled molecules to other receptors.

In some cases, GPCR agonists or antagonists can be identified (e.g., by methods described herein such as binding assays) using cells expressing a GPCR (e.g., a class B GPCR) that is amino-terminally truncated so as to prevent natural ligand binding and binding and action of molecules that might act at the natural ligand-binding site. For example, an amino-terminally truncated GPCR can be used that lacks a region corresponding to amino acid residues 66 to 75, and retains a region corresponding to amino acid residues 357 to 366, of a human secretin receptor precursor as set forth in FIG. 5.

In some cases, a Biacore (Uppsala, Sweden) system based on surface plasmon resonance can be used to identify a GPCR agonist. A region of a GPCR, such as a region that is located between the beginning of TMD 6 and the end of TMD 7, or a region that extends from the last few amino acid residues of TMD 6 to the first few amino acid residues of the loop between TMD 6 and 7, can be immobilized on a sensor surface, and any molecular interaction with the immobilized polypeptide can be monitored as a change in the intensity of surface plasmon resonance. For example, interactions with small molecules, polypeptides, or peptidomimetics can be monitored. The specificity, affinity, and kinetics of an interaction can be determined. Once a molecule is identified that interacts with a region of a GPCR, the molecule can be tested as described herein (e.g., using a cAMP assay) to determine whether or not the molecule is a GPCR agonist.

In some cases, a computational method can be used to identify a molecule that interacts with a GPCR. In particular, a computational method can be used to model interactions between molecules and particular amino acids or amino acid regions of a GPCR, such as a region corresponding to amino acid residues 357 to 366 of human secretin receptor precursor as set forth in FIG. 5 and SEQ ID NO:14. In some cases, a computational method can be used to model interactions between a GPCR and small molecules in a physical or virtual library. Interactions between a GPCR and candidate molecules identified computationally can be tested using any method, including methods described herein.

Molecules having GPCR agonist activity can be used to treat mammals (e.g., humans, dogs, horses, and cats) having a condition that can be ameliorated by stimulation of a GPCR, such as a condition associated with middle age to old age. For example, a GPCR agonist can be used to treat mammals having type 2 diabetes and/or obesity. In some cases, a GPCR agonist can be used to treat mammals having osteoporosis. In some cases, a GPCR agonist can be used to treat mammals having inflammatory bowel disease and short bowel syndrome.

A GPCR agonist can be formulated for administration by any route. For example, a GPCR agonist can be formulated for oral administration or administration by injection (e.g., subcutaneous, intravenous, or intramuscular injection). Any method can be used to formulate a composition containing a GPCR agonist provided herein. For example, common formulation mixing and preparation techniques can be used to make a composition having the components described herein. In addition, the compositions provided herein can be in any form. For example, a composition provided herein can be in the form of a solid, liquid, and/or aerosol including, without limitation, powders, crystalline substances, gels, solutions, suspensions, partial liquids, sprays, pills, capsules, tablets, and gelcaps. Typically, a composition containing one or more than one GPCR agonist can be prepared for oral administration by mixing the components with one or more of the following: a filler, a binder, a disintegrator, a lubricant, and a coloring agent. Lactose, corn starch, sucrose, glucose, sorbitol, crystalline cellulose, silicon dioxide, or the like can be used as the filler. Polyvinyl alcohol, polyvinyl ether, ethyl cellulose, methyl cellulose, gelatin, hydroxypropyl cellulose, hydroxypropylmethyl cellulose, calcium citrate, dextrin, or pectin can be used as the binder. Magnesium stearate, talc, polyethylene glycol, silica, or hardened plant oil can be used as the lubricant. A pharmaceutically acceptable coloring agent can be used as the coloring agent.

A composition (e.g., pill or tablet) containing one or more than one GPCR agonist can be formulated to contain additional components such as pharmaceutically acceptable aqueous vehicles or pharmaceutically acceptable solid vehicles. Examples of pharmaceutically acceptable aqueous vehicles include, without limitation, saline, water, and acetic acid. Typically, pharmaceutically acceptable aqueous vehicles are sterile. Any well known pharmaceutically acceptable material such as gelatin and cellulose derivatives can be used as a pharmaceutically acceptable solid vehicle. In addition, a pharmaceutically acceptable solid vehicle can be a solid carrier including, without limitation, starch, sugar, or bentonite. Further, a composition can be made using conventional procedures that employ solid carriers, lubricants, and the like.

A capsule, tablet, or particle containing one or more than one GPCR agonist can be covered with an enteric coating (e.g., a polymer) effective for shielding the capsule, tablet, or particle from digestion during transit through the upper portions of the digestive tract. An enteric coating can be a cellulose- or acrylic-based coating. An example of a cellulose-based coating is a cellulose acetate phthalate (CAP) coating, such as Aquacoat (FMC BioPolymer, Philadelphia, Pa.). A coating can dissolve when it reaches the neutral pH of the upper small intestine.

In some cases, a composition containing one or more than one GPCR agonist can be formulated for oral administration using a microencapsulation technique. For example one or more than one GPCR agonist can be mixed with a stabilizing agent in an aqueous solution. The solution can be coated onto edible beads, e.g., nonpareils, and microencapsulated with a water emulsifiable enteric coating composition. The stabilizing agent can be any agent that protects a therapeutic polypeptide from denaturation during the encapsulation process. See, for example, U.S. Pat. No. 6,613,332. In some cases, microcapsules of chitosan-alginate modified with excipients such as HPMCAS, talc, microcrystalline cellulose, polymethacrylates, and/or pectins can be used to formulate a composition provided herein. In addition, carriers including, without limitation, hydrogels, nanoparticles, and liposomes, can be used.

In some cases, a composition containing one or more than one GPCR agonist can be formulated for parenteral administration, particularly in the form of liquid solutions or suspensions in aqueous physiological buffer solutions. Formulations for parenteral administration may contain excipients suitable for injection into a mammal (e.g., a human), including sterile water or saline, oils of vegetable origin, hydrogenated naphthalenes, ammonium acetate, benzalkonium chloride, benzethonium chloride, benzyl alcohol, Brij 35, Brij 97, calcium gluceptate, chlorobutanol, polyoxyethylated castor oil, deoxycholate, citric acid monohydrate, diethanolamine, ethanol, gamma cyclodextrin, glycerin, lactobionic acid, lysine, magnesium chloride, mannitol, methylparaben, polyalkylene glycols, polyethylene glycol, PEG 1000, PEG 300, PEG 3350, PEG 400, PEG 600, polyethylene glycol 40 stearate, poloxamer 188, poloxamer 237, poloxamer 338, poloxamer 407, polyoxyethylene 100 stearate, polyoxyethylene 40 stearate, polyoxyethylene 50 stearate, polysorbate 20, polysorbate 80, povidone, propylene glycol, saccharin sodium, sodium acetate, sodium citrate dehydrate, sodium deoxycholate, sodium benzoate, and sodium tartrate. In particular, biocompatible, biodegradable lactide polymer, lactide/glycolide copolymer, or polyoxyethylene-polyoxypropylene copolymers are examples of excipients for controlling the release of a compound in vivo. Other suitable parenteral delivery systems include ethylene-vinyl acetate copolymer particles, implantable devices such as osmotic pumps or other implantable infusion systems, mucosal delivery systems, and liposomes.

In some cases, a composition containing one or more than one GPCR agonist can be a powder (e.g., a lyophilized powder). Such a powder can be reconstituted prior to administration (e.g., parenteral administration). The powder can be reconstituted with a diluent, such as a preservative-free diluent. Examples of such diluents include 5% Dextrose Injection (D5W), USP; 0.9% Sodium Chloride Injection, USP; 5% Dextrose and 0.45% Sodium Chloride Injection, USP; and 5% Dextrose and 0.2% Sodium Chloride Injection, USP.

A composition provided herein, e.g., a composition containing one or more than one GPCR agonist, can be administered to a mammal in any amount, at any frequency, and for any duration effective to achieve a desired outcome. A desired outcome can include a reduction in a symptom of type 2 diabetes (e.g., hyperglycemia), obesity (e.g., high body mass index), and/or osteoporosis (e.g., bone fractures). An effective amount of a composition can be any amount that achieves a desired result in a mammal without producing significant toxicity to the mammal. If a particular mammal fails to respond to a particular amount of a composition, then the amount can be increased by, for example, 1.1, 1.2, 1.3, 1.4, 1.5, 1.6, 1.7, 1.8, 1.9, 2, 3, 4, 5, 6, 7, 8, 9, or 10 fold. After receiving an increased amount of a composition, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple GPCR agonists within a single composition, route of administration, and severity of the condition may require an increase or decrease in the actual effective amount administered.

The frequency of administration can be any frequency that achieves a desired outcome in a mammal without producing significant toxicity to the mammal. For example, the frequency of administration can be from about four times a day to about once every other month, or from about once a day to about once a month, or from about once every other day to about once a week. In addition, the frequency of administration can remain constant or can be variable during the duration of treatment. As with the effective amount, various factors can influence the actual frequency of administration used for a particular application. For example, the effective amount, duration of treatment, use of multiple GPCR agonists within a single composition, route of administration, and severity of the condition may require an increase or decrease in administration frequency.

An effective duration of administration can be any duration that achieves a desired result in a mammal without producing significant toxicity to the mammal. The effective duration can vary from several days to several weeks, months, or years. In general, the effective duration can range from several days to several months. Once the administrations are stopped, however, a condition may return. Thus, the effective duration for preventing the return of a condition can be in some cases for as long as an individual mammal is alive. Typically, an effective duration can range from about one to two weeks to about 36 months. Again, prophylactic treatments can be typically longer in duration and can last throughout an individual mammal's lifetime. Multiple factors can influence the actual effective duration used for a particular treatment or prevention regimen. For example, an effective duration can vary with the frequency of administration, amount administered, use of multiple GPCR agonists within a single composition, route of administration, and severity of the condition. After administering a composition provided herein to a mammal, the mammal can be monitored to determine whether or not the desired result has been achieved. For example, a mammal's baseline body mass index, blood glucose level, and/or bone density before treatment can be compared to the corresponding level at various time points after treatment (e.g., one or more weeks, months, or years after treatment). A decrease in body mass index and/or blood glucose level relative to the baseline level is indicative of an improvement in obesity and/or type 2 diabetes. An increase in bone density, or a reduction in the number of bone fractures, relative to the baseline level can indicate an improvement in osteoporosis.

The invention will be further described in the following examples, which do not limit the scope of the invention described in the claims.

### EXAMPLES

### Example 1

### GPCR Agonist Activity of Polypeptides Corresponding to the Receptor Amino-Terminal Domain

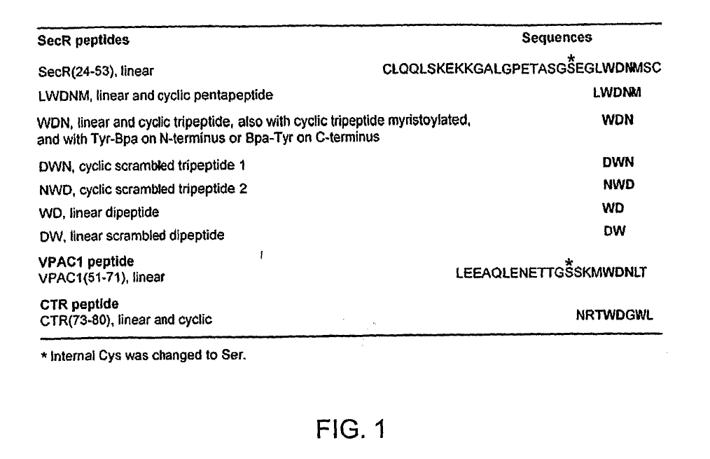

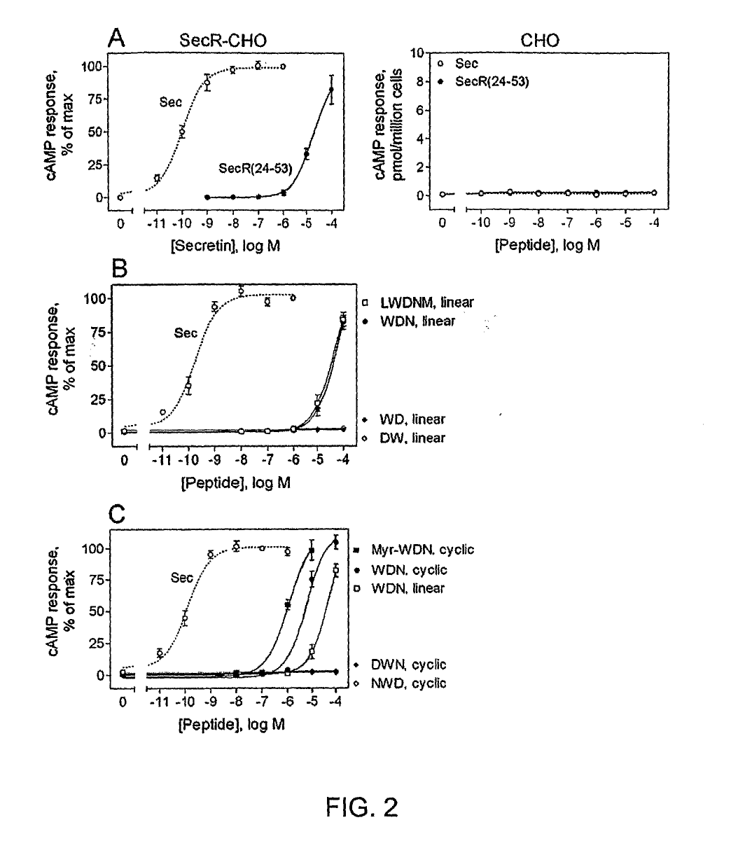

It was determined whether a synthetic polypeptide corresponding to the region of the secretin receptor amino terminus between residues 24 and 53 (FIG. 1), which shares no homology with any part of the natural agonist polypeptide, could act as an endogenous agonist ligand. Indeed, the polypeptide was a weakly potent agonist (FIG. 2A). As presented in FIG. 2A, the polypeptide stimulated cAMP accumulation in a Chinese hamster ovary (CHO) cell line expressing the secretin receptor. The cAMP response was dependent on the concentration of the polypeptide. Despite its low potency, the efficacy of the polypeptide was similar to that of secretin (FIG. 2A). The polypeptide had no effect on cAMP levels in the parental CHO cell line, even at concentrations as high as 100 μM (FIG. 2A). The polypeptide also did not affect cAMP levels in CHO cell lines engineered to express high concentrations of a structurally-distinct Class A G protein-coupled cholecystokinin receptor.

To define the shortest sequence responsible for this effect, a series of synthetic polypeptides (FIG. 1) were tested for their ability to stimulate cAMP accumulation. Agonist activity was maintained when the polypeptide was reduced in length from 30 residues to five residues in the region focused on a highly conserved aspartate residue (FIG. 2B). Even a polypeptide only three residues in length, including the conserved aspartate, stimulated cAMP accumulation. However, when the polypeptide was further shortened to a dipeptide, no agonist activity was observed.

The three-residue sequence was formed into a constrained loop using a diaminopropionic acid linker across the ends of the polypeptide. The potency of the three-residue sequence in the context of the constrained loop was enhanced relative to that of the linear polypeptide (FIG. 2C). In addition, the cyclic polypeptide maintained full efficacy. It is noteworthy that scrambling the sequence of the three residues eliminated all agonist activity (FIG. 2C). The potency of the cyclic polypeptide was further enhanced by fatty acid acylation (FIG. 2C).

### Example 2

### Photoaffinity Labeling

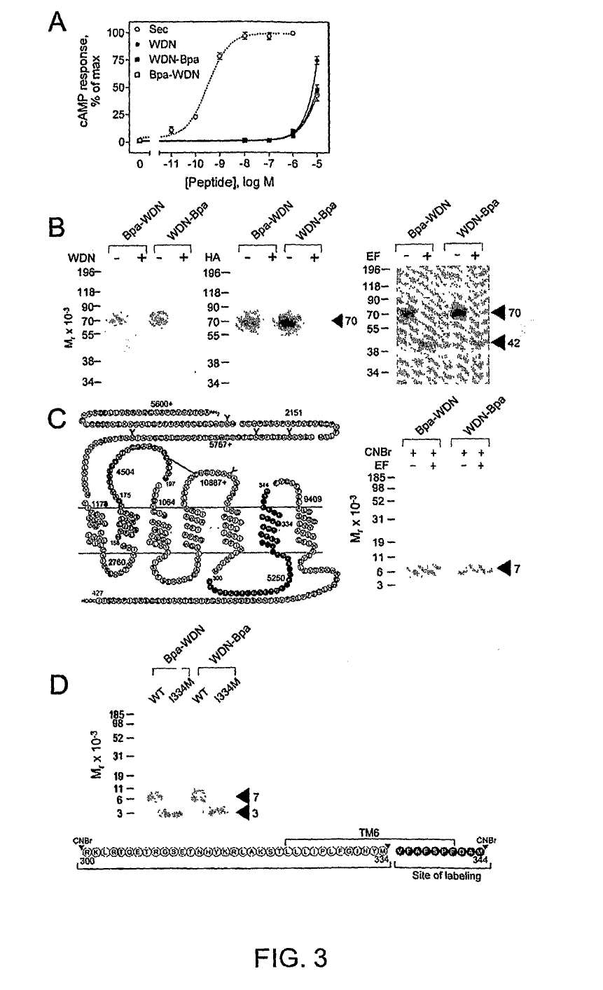

The cyclic polypeptide was modified by attaching a photolabile benzoyl-phenylalanine and a radioiodinatable tyrosine residue to the amino terminus or the carboxy terminus. The cyclic polypeptides containing both photoprobes on either end maintained their biological activity (FIG. 3A). Both cyclic polypeptides were able to covalently label a hemagglutinin (HA)-tagged secretin receptor in a saturable and specific manner, as demonstrated by autoradiography of SDS-polyacrylamide gels used to separate the membrane proteins after immunoprecipitation with anti-HA antibody (FIG. 3B). Deglycosylation of the labeled band using endoglycosidase F yielded the expected mass of the core receptor protein, confirming its identity (FIG. 3B). Further identification of the site of covalent attachment using cyanogen bromide cleavage of the labeled native and deglycosylated receptor bands yielded a mass of approximately 7 KDa (FIG. 3C). Based on the non-glycosylated nature and apparent mass of the cyanogen bromide fragments of this receptor covalently bound to this ligand (1154 Da), the most likely candidate was the fragment extending from the third intracellular loop, through transmembrane segment six, to the third extracellular loop. Using a previously-constructed receptor mutant in which the isoleucine residue in position 334 within this cyanogen bromide fragment was changed to a methionine to produce an additional site of cyanogen bromide cleavage (17), a clear shift in the electrophoretic migration of the labeled fragment was observed (FIG. 3C). This provided definitive evidence that the region covalently labeled with both probes was a decapeptide extending between residues 335 and 344 of transmembrane segment six and the beginning of its extracellular loop.

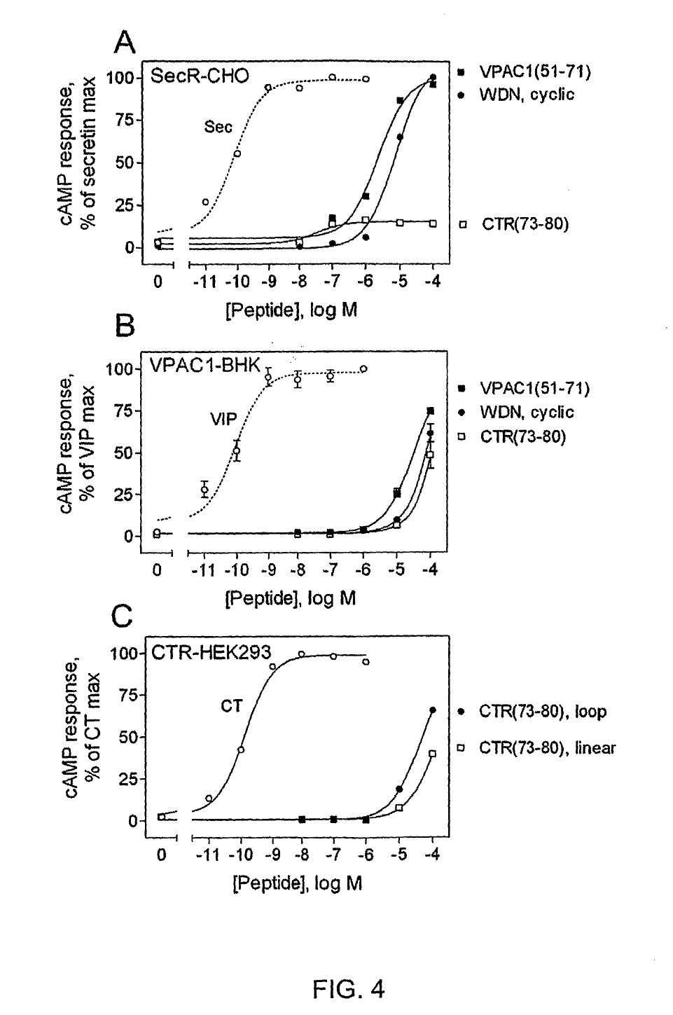

Polypeptides representing the analogous region of the amino terminus of the Class B G protein-coupled calcitonin and VPAC1 receptors (FIG. 1) were also synthesized. Each of these polypeptides also possessed low potency agonist activity at their respective receptors (FIG. 4). The agonist activity of these endogenous polypeptides was not limited to the corresponding receptors. The polypeptides were also active at other structurally-related family members (FIG. 4).

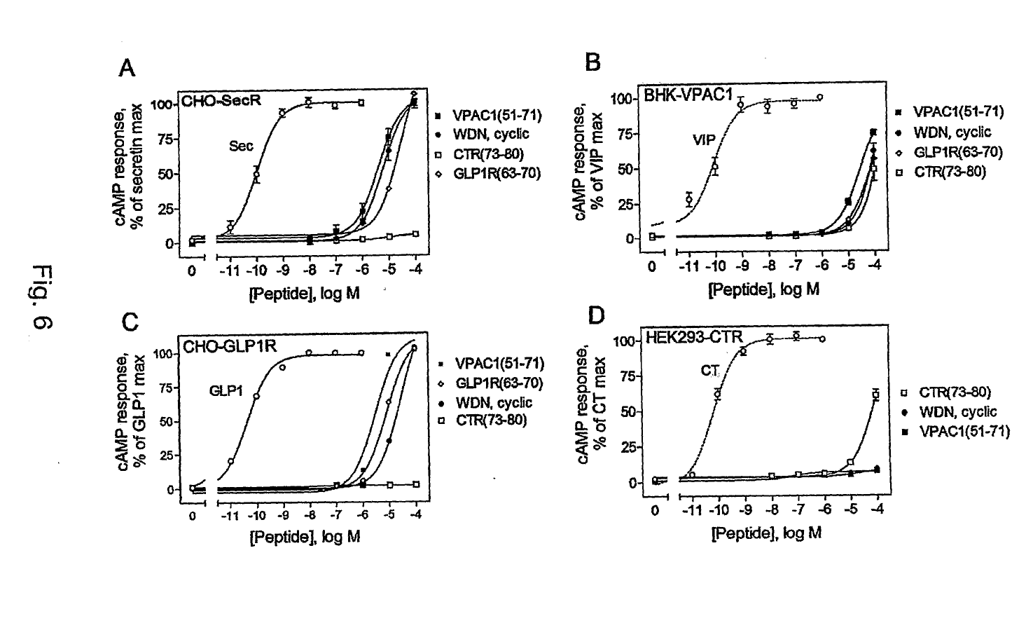

Additional experiments were performed and curves were regenerated (FIG. 6), as described herein and as described elsewhere (Dong et al., Mol. Pharmacol., 70(1):206-13 (2006)). A polypeptide (GLP1R(63-70)) corresponding to amino acid residues 63 to 70 of the human GLP1R (SEQ ID NO:15) was active as an agonist at the human GLP1R, the rat secretin receptor (SecR), and the rat VPAC1 receptor. The potency of the GLP1R(63-70) polypeptide was similar to that of the WDN tripeptide at the SecR and VPAC1 receptors (FIG. 6, Panels A-B). The WDN tripeptide and the VPAC1(51-71) polypeptide (SEQ ID NO:4) were active as agonists at the human GLP1R receptor, with potencies similar to that of the GLP1R(63-70) polypeptide. Each polypeptide used to generate the data presented in FIG. 6 was a cyclic polypeptide, except for the VPAC1(51-71) polypeptide. Linear versions of each polypeptide were also tested and found to behave in a similar manner as the corresponding cyclic polypeptides, with similar potencies and efficacies.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of stimulating a G protein-coupled receptor, said method comprising contacting said receptor with an agonist, wherein said agonist binds to a region of said receptor that is between the beginning of transmembrane domain 6 and the end of transmembrane domain 7 of said receptor.

2. The method of claim 1, wherein said G protein-coupled receptor is a class B G protein-coupled receptor.

3. The method of claim 1, wherein said G protein-coupled receptor is a human receptor.

4. The method of claim 1, wherein said G protein-coupled receptor is a calcitonin receptor, a vasoactive intestinal peptide receptor 1, a glucagon-like peptide 1 receptor, or a secretin receptor.

5. The method of claim 1, wherein said agonist is a polypeptide comprising less than 30 amino acids.

6. The method of claim 1, wherein said agonist is a polypeptide comprising less than six amino acids.

7. The method of claim 1, wherein said agonist is a polypeptide comprising less four amino acids.

8. The method of claim 1, wherein said agonist is a cyclic polypeptide.

9. The method of claim 1, wherein said agonist comprises less than 30 amino acids and comprises an amino acid sequence located between the second and third conserved cysteine residues of said G protein-coupled receptor.

10. The method of claim 1, wherein said region comprises about 10 amino acids.

11. The method of claim 1, wherein at least a portion of said region is within the loop between said transmembrane domain 6 and said transmembrane domain 7.

12. The method of claim 1, wherein said agonist comprises a tryptophan-aspartate-asparagine sequence or a structure that is conformationally comparable to said sequence.

13. A method for identifying an agonist of a G protein-coupled receptor, said method comprising:
(a) obtaining a test agent comprising a tryptophan-aspartate-asparagine sequence or a structure that is conformationally comparable to said sequence,
(b) contacting a cell comprising a G protein-coupled receptor with said test agent, and
(c) determining whether or not said test agent activated said G protein-coupled receptor, wherein the presence of said activation indicates that said test agent is an agonist of said G protein-coupled receptor.

14. The method of claim 13, wherein said G protein-coupled receptor is a class B G protein-coupled receptor.

15. The method of claim 13, wherein said G protein-coupled receptor is a human receptor.

16. The method of claim 13, wherein said G protein-coupled receptor is a calcitonin receptor, a vasoactive intestinal peptide receptor 1, a glucagon-like peptide 1 receptor, or a secretin receptor.

17. The method of claim 13, wherein said test agent is a polypeptide comprising less than 30 amino acids.

18. The method of claim 13, wherein said test agent is a polypeptide comprising less than 10 amino acids.

19. The method of claim 13, wherein said test agent is a polypeptide comprising less than 5 amino acids.

20. The method of claim 13, wherein said test agent is a non-polypeptide molecule.

21. The method of claim 13, wherein said cell expresses the secretin receptor.

22. The method of claim 21, wherein said cell is a Chinese hamster ovary cell.

23. The method of claim 13, wherein said determining step comprises a cAMP assay.

24. A substantially pure polypeptide less than 50 amino acid residues in length, wherein said polypeptide is capable of binding to a region of a G protein-coupled receptor that is between the beginning of transmembrane domain 6 and the end of transmembrane domain 7 of said receptor.

25. The polypeptide of claim 24, wherein said polypeptide is less than 40 amino acid residues in length.

26. The polypeptide of claim 24, wherein said polypeptide is less than 30 amino acid residues in length.

27. The polypeptide of claim 24, wherein said polypeptide is less than 20 amino acid residues in length.

28. The polypeptide of claim 24, wherein said polypeptide is less than 10 amino acid residues in length.

29. The polypeptide of claim 24, wherein said polypeptide is less than 5 amino acid residues in length.

30. The polypeptide of claim 24, wherein said polypeptide comprises an amino acid sequence selected from the group consisting of SEQ ID NO:1, SEQ ID NOs:3-4, SEQ ID NO:6, and SEQ ID NOs:22-32.

31. The polypeptide of claim 30, wherein said polypeptide is cyclic.

32. The polypeptide of claim 30, wherein said polypeptide comprises a diaminopropionic acid at the amino terminus and an aspartic acid residue at the carboxyl terminus, and wherein said diaminopropionic acid is linked to said aspartic acid to form a cyclic polypeptide.

33. A method for identifying an agonist of a G protein-coupled receptor, said method comprising:
(a) contacting a cell comprising a G protein-coupled receptor lacking an extracellular portion of the amino terminus of said receptor with a test agent, and
(b) determining whether or not said test agent activated said G protein-coupled receptor, wherein the presence of said activation indicates that said test agent is an agonist of said G protein-coupled receptor.


SEQUENCE LISTING (32 sequences)

SEQ ID NO: 1 - protein, 30 aa, Artificial Sequence
  modified rattus norvegicus peptide
CLQQLSKEKKGALGPETASGSEGLWDNMSC

SEQ ID NO: 2 - protein, 449 aa, Rattus norvegicus
MLSTMRPRLSLLLLRLLLLTKAAHTVGVPPRLCDVRRVLLEERAHCLQQLSKEKKGALGPETASGCEGLWDNMSCWPSSAPARTVEVQCPKFLLMLSNKNGSLFRNCTQDGWSETFPRPDLACGVNINNSFNERRHAYLLKLKVMYTVGYSSSLAMLLVALSILCSFRRLHCTRNYIHMHLFVSFILRALSNFIKDAVLFSSDDVTYCDAHKVGCKLVMIFFQYCIMANYAWLLVEGLYLHTLLAISFFSERKYLQAFVLLGWGSPAIFVALWAITRHFLENTGCWDINANASVWWVIRGPVILSILINFIFFINILRILMRKLRTQETRGSETNHYKRLAKSTLLLIPLFGIHYIVFAFSPEDAMEVQLFFELALGSFQGLVVAVLYCFLNGEVQLEVQKKWRQWHLQEFPLRPVAFNNSFSNATNGPTHSTKASTEQSRSIPRASII

SEQ ID NO: 3 - protein, 5 aa, Rattus norvegicus
LWDNM

SEQ ID NO: 4 - protein, 21 aa, Artificial Sequence
  modified rattus norvegicus peptide
LEEAQLENETTGSSKMWDNLT

SEQ ID NO: 5 - protein, 459 aa, Rattus norvegicus
MRPPSPPHVRWLCVLAGALACALRPAGSQAASPQHECEYLQLIEIQRQQCLEEAQLENETTGCSKMWDNLTCWPTTPRGQAVVLDCPLIFQLFAPIHGYNISRSCTEEGWSQLEPGPYHIACGLNDRASSLDEQQQTKFYNTVKTGYTIGYSLSLASLLVAMAILSLFRKLHCTRNYIHMHLFMSFILRATAVFIKDMALFNSGEIDHCSEASVGCKAAVVFFQYCVMANFFWLLVEGLYLYTLLAVSFFSERKYFWGYILIGWGVPSVFITIWTVVRIYFEDFGCWDTIINSSLWWIIKAPILLSILVNFVLFICIIRILVQKLRPPDIGKNDSSPYSRLAKSTLLLIPLFGIHYVMFAFFPDNFKAQVKMVFELVVGSFQGFVVAILYCFLNGEVQAELRRKWRRWHLQGVLGWSSKSQHPWGGSNGATCSTQVSMLTRVSPSARRSSSFQAEVSLV

SEQ ID NO: 6 - protein, 8 aa, Homo sapiens
NRTWDGWL

SEQ ID NO: 7 - protein, 474 aa, Homo sapiens
MRFTFTSRCLALFLLLNHPTPILPAFSNQTYPTIEPKPFLYVVGRKKMMDAQYKCYDRMQQLPAYQGEGPYCNRTWDGWLCWDDTPAGVLSYQFCPDYFPDFDPSEKVTKYCDEKGVWFKHPENNRTWSNYTMCNAFTPEKLKNAYVLYYLAIVGHSLSIFTLVISLGIFVFFRSLGCQRVTLHKNMFLTYILNSMIIIIHLVEVVPNGELVRRDPVSCKILHFFHQYMMACNYFWMLCEGIYLHTLIVVAVFTEKQRLRWYYLLGWGFPLVPTTIHAITRAVYFNDNCWLSVETHLLYIIHGPVMAALVVNFFFLLNIVRVLVTKMRETHEAESHMYLKAVKATMILVPLLGIQFVVFPWRPSNKMLGKIYDYVMHSLIHFQGFFVATIYCFCNNEVQTTVKRQWAQFKIQWNQRWGRRPSNRSARAAAAAAEAGDIPIYICHQELRNEPANNQGEESAEIIPLNIIEQESSA

SEQ ID NO: 8 - protein, 427 aa, Rattus norvegicus
AHTVGVPPRLCDVRRVLLEERAHCLQQLSKEKKGALGPETASGCEGLWDNMSCWPSSAPARTVEVQCPKFLLMLSNKNGSLFRNCTQDGWSETFPRPDLACGVNINNSFNERRHAYLLKLKVMYTVGYSSSLAMLLVALSILCSFRRLHCTRNYIHMHLFVSFILRALSNFIKDAVLFSSDDVTYCDAHKVGCKLVMIFFQYCIMANYAWLLVEGLYLHTLLAISFFSERKYLQAFVLLGWGSPAIFVALWAITRHFLENTGCWDINANASVWWVIRGPVILSILINFIFFINILRILMRKLRTQETRGSETNHYKRLAKSTLLLIPLFGIHYIVFAFSPEDAMEVQLFFELALGSFQGLVVAVLYCFLNGEVQLEVQKKWRQWHLQEFPLRPVAFNNSFSNATNGPTHSTKASTEQSRSIPRASII

SEQ ID NO: 9 - protein, 22 aa, Rattus norvegicus
MLSTMRPRLSLLLLRLLLLTKA

SEQ ID NO: 10 - protein, 45 aa, Rattus norvegicus
RKLRTQETRGSETNHYKRLAKSTLLLIPLFGIHYMVFAFSPEDAM

SEQ ID NO: 11 - protein, 438 aa, Homo sapiens
MRTLLPPALLTCWLLAPVNSIHPECRFHLEIQEEETKCAELLRSQTEKHKACSGVWDNITCWRPANVGETVTVPCPKVFSNFYSKAGNISKNCTSDGWSETFPDFVDACGYSDPEDESKITFYILVKAIYTLGYSVSLMSLATGSIILCLFRKLHCTRNYIHLNLFLSFILRAISVLVKDDVLYSSSGTLHCPDQPSSWVGCKLSLVFLQYCIMANFFWLLVEGLYLHTLLVAMLPPRRCFLAYLLIGWGLPTVCIGAWTAARLYLEDTGCWDTNDHSVPWWVIRIPILISIIVNFVLFISIIRILLQKLTSPDVGGNDQSQYKRLAKSTLLLIPLFGVHYMVFAVFPISISSKYQILFELCLGSFQGLVVAVLYCFLNSEVQCELKRKWRSRCPTPSASRDYRVCGSSFSRNGSEGALQFHRGSRAQSFLQTETSVI

SEQ ID NO: 12 - protein, 468 aa, Homo sapiens
MAGVVHVSLAALLLLPMAPAMHSDCIFKKEQAMCLEKIQRANELMGFNDSSPGCPGMWDNITCWKPAHVGEMVLVSCPELFRIFNPDQVWETETIGESDFGDSNSLDLSDMGVVSRNCTEDGWSEPFPHYFDACGFDEYESETGDQDYYYLSVKALYTVGYSTSLVTLTTAMVILCRFRKLHCTRNFIHMNLFVSFMLRAISVFIKDWILYAEQDSNHCFISTVECKAVMVFFHYCVVSNYFWLFIEGLYLFTLLVETFFPERRYFYWYTIIGWGTPTVCVTVWATLRLYFDDTGCWDMNDSTALWWVIKGPVVGSIMVNFVLFIGIIVILVQKLQSPDMGGNESSIYLRLARSTLLLIPLFGIHYTVFAFSPENVSKRERLVFELGLGSFQGFVVAVLYCFLNGEVQAEIKRKWRSWKVNRYFAVDFKHRHPSLASSGVNGGTQLSILSKSSSQIRMSGLPADNLAT

SEQ ID NO: 13 - protein, 457 aa, Homo sapiens
MRPPSPLPARWLCVLAGALAWALGPAGGQAARLQEECDYVQMIEVQHKQCLEEAQLENETIGCSKMWDNLTCWPATPRGQVVVLACPLIFKLFSSIQGRNVSRSCTDEGWTHLEPGPYPIACGLDDKAASLDEQQTMFYGSVKTGYTIGYGLSLATLLVATAILSLFRKLHCTRNYIHMHLFISFILRAAAVFIKDLALFDSGESDQCSEGSVGCKAAMVFFQYCVMANFFWLLVEGLYLYTLLAVSFFSERKYFWGYILIGWGVPSTFTMVWTIARIHFEDYGCWDTINSSLWWIIKGPILTSILVNFILFICIIRILLQKLRPPDIRKSDSSPYSRLARSTLLLIPLFGVHYIMFAFFPDNFKPEVKMVFELVVGSFQGFVVAILYCFLNGEVQAELRRKWRRWHLQGVLGWNPKYRHPSGGSNGATCSTQVSMLTRVSPGARRSSSFQAEVSLV

SEQ ID NO: 14 - protein, 440 aa, Homo sapiens
MRPHLSPPLQQLLLPVLLACAAHSTGALPRLCDVLQVLWEEQDQCLQELSREQTGDLGTEQPVPGCEGMWDNISCWPSSVPGRMVEVECPRFLRMLTSRNGSLFRNCTQDGWSETFPRPNLACGVNVNDSSNEKRHSYLLKLKVMYTVGYSSSLVMLLVALGILCAFRRLHCTRNYIHMHLFVSFILRALSNFIKDAVLFSSDDVTYCDPHRAGCKLVMVLFQYCIMANYSWLLVEGLYLHTLLAISFFSERKYLQGFVAFGWGSPAIFVALWAIARHFLEDVGCWDINANASIWWIIRGPVILSILINFILFINILRILMRKLRTQETRGNEVSHYKRLARSTLLLIPLFGIHYIVFAFSPEDAMEIQLFFELALGSFQGLVVAVLYCFLNGEVQLEVQKKWQQWHLREFPLHPVASFSNSTKASHLEQSQGTCRTSII

SEQ ID NO: 15 - protein, 463 aa, Homo sapiens
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYIIYTVGYALSFSALVIASAILLGFRHLHCTRNYIHLNLFASFILRALSVFIKDAALKWMYSTAAQQHQWDGLLSYQDSLSCRLVFLLMQYCVAANYYWLLVEGVYLYTLLAFSVFSEQWIFRLYVSIGWGVPLLFVVPWGIVKYLYEDEGCWTRNSNMNYWLIIRLPILFGIGVNFLIFVRVICIVVSKLKANLMCKTDIKCRLAKSTLTLIPLLGTHEVIFAFVMDEHARGTLRFIKLFTELSFTSFQGLMVAILYCFVNNEVQLEFRKSWERWRLEHLHIQRDSSMKPLKCPTSSLSSGATAGSSMYTATCQASCS

SEQ ID NO: 16 - protein, 553 aa, Homo sapiens
MKLGSSRAGPGRGSAGLLPGVHELPMGIPAPWGTSPLSFHRKCSLWAPGRPFLTLVLLVSIKQVTGSLLEETTRKWAQYKQACLRDLLKEPSGIFCNGTFDQYVCWPHSSPGNVSVPCPSYLPWWSEESSGRAYRHCLAQGTWQTIENATDIWQDDSECSENHSFKQNVDRYALLSTLQLMYTVGYSFSLISLFLALTLLLFLRKLHCTRNYIHMNLFASFILRTLAVLVKDVVFYNSYSKRPDNENGWMSYLSEMSTSCRSVQVLLHYFVGANYLWLLVEGLYLHTLLEPTVLPERRLWPRYLLLGWAFPVLFVVPWGFARAHLENTGCWTTNGNKKIWWIIRGPMMLCVTVNFFIFLKILKLLISKLKAHQMCFRDYKYRLAKSTLVLIPLLGVHEILFSFITDDQVEGFAKLIRLFIQLTLSSFHGFLVALQYGFANGEVKAELRKYWVRFLLARHSGCRACVLGKDFRFLGKCPKKLSEGDGAEKLRKLQPSLNSGRLLHLAMRGLGELGAQPQQDHARWPRGSSLSECSEGDVTMANTMEEILEESEI

SEQ ID NO: 17 - protein, 593 aa, Homo sapiens
MGTARIAPGLALLLCCPVLSSAYALVDADDVMTKEEQIFLLHRAQAQCEKRLKEVLQRPASIMESDKGWTSASTSGKPRKDKASGKLYPESEEDKEAPTGSRYRGRPCLPEWDHILCWPLGAPGEVVAVPCPDYIYDFNHKGHAYRRCDRNGSWELVPGHNRTWANYSECVKFLTNETREREVFDRLGMIYTVGYSVSLASLTVAVLILAYFRRLHCTRNYIHMHLFLSFMLRAVSIFVKDAVLYSGATLDEAERLTEEELRAIAQAPPPPATAAAGYAGCRVAVTFFLYFLATNYYWILVEGLYLHSLIFMAFFSEKKYLWGFTVFGWGLPAVFVAVWVSVRATLANTGCWDLSSGNKKWIIQVPILASIVLNFILFINIVRVLATKLRETNAGRCDTRQQYRKLLKSTLVLMPLFGVHYIVFMATPYTEVSGTLWQVQMHYEMLFNSFQGFFVAIIYCFCNGEVQAEIKKSWSRWTLALDFKRKARSGSSSYSYGPMVSHTSVTNVGPRVGLGLPLSPRLLPTATTNGHPQLPGHAKPGTPALETLETTPPAMAAPKDDGFLNGSCSGLDEEASGPERPPALLQEEWETVM

SEQ ID NO: 18 - protein, 550 aa, Homo sapiens
MAGLGASLHVWGWLMLGSCLLARAQLDSDGTITIEEQIVLVLKAKVQCELNITAQLQEGEGNCFPEWDGLICWPRGTVGKISAVPCPPYIYDFNHKGVAFRHCNPNGTWDFMHSLNKTWANYSDCLRFLQPDISIGKQEFFERLYVMYTVGYSISFGSLAVAILIIGYFRRLHCTRNYIHMHLFVSFMLRATSIFVKDRVVHAHIGVKELESLIMQDDPQNSIEATSVDKSQYIGCKIAVVMFIYFLATNYYWILVEGLYLHNLIFVAFFSDTKYLWGFILIGWGFPAAFVAAWAVARATLADARCWELSAGDIKWIYQAPILAAIGLNFILFLNTVRVLATKIWETNAVGHDTRKQYRKLAKSTLVLVLVFGVHYIVFVCLPHSFTGLGWEIRMHCELFFNSFQGFFVSIIYCYCNGEVQAEVKKMWSRWNLSVDWKRTPPCGSRRCGSVLTTVTHSTSSQSQVAASTRMVLISGKAAKIASRQPDSHITLPGYVWSNSEQDCLPHSFHEETKEDSGRQGDDILMEKPSRPMESNPDTEGCQGETEDVL

SEQ ID NO: 19 - protein, 411 aa, Homo sapiens
MDAALLHSLLEANCSLALAEELLLDGWGPPLDPEGPYSYCNTTLDQIGTCWPRSAAGALVERPCPEYFNGVKYNTTRNAYRECLENGTWASKINYSQCEPILDDKQRKYDLHYRIALVVNYLGHCVSVAALVAAFLLFLALRSIRCLRNVIHWNLITTFILRNVMWFLLQLVDHEVHESNEVWCRCITTIFNYFVVTNFFWMFVEGCYLHTAIVMTYSTERLRKCLFLFIGWCIPFPIIVAWAIGKLYYENEQCWFGKEPGDLVDYIYQGPIILVLLINFVFLFNIVRILMTKLRASTTSETIQYRKAVKATLVLLPLLGITYMLFFVNPGEDDLSQIMFIYFNSFLQSFQGFFVSVFYCFFNGEVRSAVRKRWHRWQDHHSLRVPMARAMSIPTSPTRISFHSIKQTAAV

SEQ ID NO: 20 - protein, 415 aa, Homo sapiens
MGGHPQLRLVKALLLLGLNPVSASLQDQHCESLSLASNISGLQCNASVDLIGTCWPRSPAGQLVVRPCPAFFYGVRYNTTNNGYRECLANGSWAARVNYSECQEILNEEKKSKVHYHVAVIINYLGHCISLVALLVAFVLFLRLRSIRCLRNIIHWNLISAFILRNATWFVVQLTMSPEVHQSNVGWCRLVTAAYNYFHVTNFFWMFGEGCYLHTAIVLTYSTDRLRKWMFICIGWGVPFPIIVAWAIGKLYYDNEKCWFGKRPGVYTDYIYQGPMILVLLINFIFLFNIVRILMTKLRASTTSETIQYRKAVKATLVLLPLLGITYMLFFVNPGEDEVSRVVFIYFNSFLESFQGFFVSVFYCFLNSEVRSAIRKRWHRWQDKHSIRARVARAMSIPTSPTRVSFHSIKQSTAV

SEQ ID NO: 21 - protein, 461 aa, Homo sapiens [sequence omitted]

SEQ ID NO: 22 - protein, 8 aa, Homo sapiens
NRTFDEYA

SEQ ID NO: 23 - protein, 5 aa, Homo sapiens
VWDNI

SEQ ID NO: 24 - protein, 5 aa, Homo sapiens
MWDNI

SEQ ID NO: 25 - protein, 5 aa, Homo sapiens
MWDNL

SEQ ID NO: 26 - protein, 5 aa, Homo sapiens
TFDEY

SEQ ID NO: 27 - protein, 5 aa, Homo sapiens
TFDQY

SEQ ID NO: 28 - protein, 5 aa, Homo sapiens
TLDQI

SEQ ID NO: 29 - protein, 5 aa, Homo sapiens
TWDGW

SEQ ID NO: 30 - protein, 5 aa, Homo sapiens
EWDHI

SEQ ID NO: 31 - protein, 5 aa, Homo sapiens
EWDGL

SEQ ID NO: 32 - protein, 5 aa, Homo sapiens
SVDLI